SEQUENCE LISTING (14 sequences)

SEQ ID NO: 1 - protein, 6 aa, synthetic construct
EGSNGY

SEQ ID NO: 2 - protein, 12 aa, synthetic construct
YDSYNSKVVLES

SEQ ID NO: 3 - protein, 15 aa, synthetic construct
CATWDGSRSYYKKLF

SEQ ID NO: 4 - protein, 7 aa, synthetic construct
TSWWSYY

SEQ ID NO: 5 - protein, 6 aa, synthetic construct
QGSDEQ

SEQ ID NO: 6 - protein, 14 aa, synthetic construct
CALGDYVLSDKLIF

SEQ ID NO: 7 - protein, 12 aa, synthetic construct
CATWDPYYKKLF

SEQ ID NO: 8 - protein, 17 aa, synthetic construct
CALDLGGRGPPPDKLIF

SEQ ID NO: 9 - DNA, 18 bp, synthetic construct
  source 1..18: mol_type = other DNA
gaaggaagtaacggctac

SEQ ID NO: 10 - DNA, 36 bp, synthetic construct
  source 1..36: mol_type = other DNA
tatgactcctacaactccaaggttgtgttggaatca

SEQ ID NO: 11 - DNA, 45 bp, synthetic construct
  source 1..45: mol_type = other DNA
tgtgccacctgggacgggtcccgaagctattataagaaactcttt

SEQ ID NO: 12 - DNA, 21 bp, synthetic construct
  source 1..21: mol_type = other DNA
acaagttggtggtcatattat

SEQ ID NO: 13 - DNA, 18 bp, synthetic construct
  source 1..18: mol_type = other DNA
cagggttctgatgaacag

SEQ ID NO: 14 - DNA, 42 bp, synthetic construct
  source 1..42: mol_type = other DNA
tgtgctcttggggactacgttctatccgataaactcatcttt

(19) United States
(12) Patent Application Publication
(10) Pub. No.: US 2026/0285936 A1
(43) Pub. Date: Sep. 24, 2026

(54) TUMOR SPECIFIC GAMMA DELTA T CELLS

(71) Applicant: UNIVERSITY HEALTH NETWORK, Toronto (CA)

(72) Inventors: Pamela Sumiko OHASHI, Toronto (CA); Albiruni RAZAK, Toronto (CA); Naoto HIRANO, Toronto (CA); Dalam LY, Toronto (CA); Scott LIEN, Toronto (CA)

(21) Appl. No.: 19/476,036

(22) PCT Filed: Apr. 16, 2024
(86) PCT No.: PCT/CA2024/050488

§ 371 Date: Oct. 16, 2025

Related U.S. Application Data
(60) Provisional application No. 63/459,683, filed on Apr. 16, 2023.

(51) Int. Cl. C07K 14/705; A61K 40/11; A61K 40/32; A61P 35/00; C07K 16/28; C12N 5/0783; C12N 15/86

(52) U.S. Cl. CPC C07K 14/7051; A61K 40/11; A61K 40/32; A61P 35/00; C07K 16/2809; C12N 5/0636; C12N 15/86; C12N 2510/00; C12N 2740/10043

(57) ABSTRACT

A novel γδ T cell receptor heterodimer is provided with application in cancer treatments.

### CROSS-REFERENCE TO RELATED APPLICATIONS AND DOCUMENTS

This application claims priority to U.S. provisional patent application No. 63/459,683 filed Apr. 16, 2023, the content of which are incorporated herein by reference. This application also comprises a sequence listing in electronic form which is also incorporated in its entirety.

### TECHNICAL FIELD

This disclosure generally relates to the field of γδ T cell receptors and T cell transfer therapies.

### BACKGROUND OF THE ART

With the breakthrough of checkpoint blockade, immunotherapies targeting PD-1/PD-L1 have had remarkable success in the clinic and are now widely used to treat a variety of malignancies¹⁻³. PD-1 blockade reinvigorates Tcf1⁺ precursor exhausted T cells (Tₚₑₓ) that replenish the pool of T cells with effector functions^{4,5}. The majority of research on T cell exhaustion and PD-1 blockade has been focused on conventional αβ T cells, and the contribution of innate-like T cells such as γδ T cells to PD-1 blockade is not clear.

γδ T cells are innate-like T cells that are generally not restricted to conventional MHC-peptide molecules. In the tumor microenvironment, γδ T cells can recognize a wide variety of molecules through TCR-dependent and independent mechanisms, including phosphoantigens presented on butyrophilins^{6,7}, MHC class I-like family members MR1⁸ and CD1c⁹, as well as stress-induced molecules like MICA¹⁰.

In conventional αβ T cells, chronic antigen stimulation leads to T cell exhaustion¹²⁻¹⁴, which can be characterized by progressive loss of effector functions¹⁵, expression of inhibitory receptors¹⁶, as well as transcriptional and epigenetic remodeling¹⁷⁻²¹. However, whether γδ T cells are a therapeutic target of PD-1 blockade has remained unclear.

### SUMMARY

In one aspect, there is disclosed a novel γδ T cell receptor heterodimer. In one embodiment, there is provided a vector comprising a nucleic acid sequence encoding a T cell receptor heterodimer, or a T cell receptor γ peptide chain capable of forming a functional T cell receptor heterodimer together with a T cell receptor δ peptide chain, and/or a T cell receptor δ peptide chain capable of forming a functional T cell receptor heterodimer together with a T cell receptor γ peptide chain, wherein the nucleic acid sequence encodes CATWDGSRSYYKKLF (SEQ ID NO: 3) as complementarity-determining region 3γ (CDR3γ) and/or CALGDYVLSDKLIF (SEQ ID NO: 6) as complementarity-determining region 3δ (CDR3δ).

In one embodiment, the vector encodes: a first polypeptide comprising three γ complementarity-determining regions (CDRs) comprising the amino acid sequences: EGSNGY (SEQ ID NO: 1) as CDR1γ, YDSYNSKVVLES (SEQ ID NO: 2) as CDR2γ and CATWDGSRSYYKKLF (SEQ ID NO: 3) as CDR3γ; and/or a second polypeptide comprising three CDRs comprising the amino acid sequences: TSWWSYY (SEQ ID NO: 4) as CDR1δ, QGSDEQ (SEQ ID NO: 5) as CDR2δ, and CALGDYVLSDKLIF (SEQ ID NO: 6) as CDR3δ.

Also provided are host cells and transduced T cells expressing a T cell receptor (TCR) as disclosed herein, and bi-specific antibodies and antibody-drug conjugates that include a T cell receptor as defined herein or a functional fragment thereof. These may be used in methods for the treatment of proliferative disease, optionally cancer, or an autoimmune disease. Aspects of the present disclosure are directed to a T cell receptor (TCR) or an antigen binding portion thereof comprising the gamma chain variable domain disclosed herein and the delta chain variable domain disclosed herein.

Many further features and combinations thereof concerning the present improvements will appear to those skilled in the art following a reading of the instant disclosure.

### DESCRIPTION OF THE DRAWINGS

FIG. 1 shows an example of γδ T cells proliferating and expanding in tumor and blood upon pembrolizumab treatment. A, Left and middle flow plots of γδ T cells in baseline and week 9 tumor biopsies from patient E-013. Right, quantification of γδ T cells in tumor biopsies in six MCC patients. B, Flow plot of PD-1 and TIGIT expression gated on γδ T cells from baseline biopsy of patient E-013 and C, E-022. D, γδ T cell frequency measured in peripheral blood. E, Flow plot of Vδ1 and Vδ2 subsets gated on γδ T cells from E-013 baseline blood. Right, summary of γδ T cell in peripheral blood at baseline and week 6. F, Proliferation of γδ T cells from blood of E-013. G, Summary of proliferating γδ T cells in blood. H, PD-1 and TIM3 expression gated on γδ T cells in E-013 baseline blood. I, Summary of TIM3+ γδ T cells in blood. Dark grey denotes partial response and complete response, light grey represents progressive disease. Dark grey dotted line is patient E-013 and light grey dotted line is E-035 (A, D, E, G).

FIG. 2 shows an example of γδ T cell proliferation and cytokine production as a result of PD-1 blockade. A, γδ T cells isolated from healthy donor peripheral blood mononuclear cells (PBMCs) were co-cultured with either the Merkel cell carcinoma (MCC) line MS-1 cells, or MS-1 cells pulsed with the phosphoantigen €-4-Hydroxy-3-methyl-but-2-enyl pyrophosphate (HMB-PP) for four hours. Human IgG4 isotype or pembrolizumab was added, and after three days, dilution of proliferation dye was measured by flow cytometry. B, Proliferation index summarized for three different healthy donors. C, Intracellular cytokines were measured by adding Brefeldin A at beginning of co-culture. D, Cytokines in supernatant were measured after three days by flow cytometric bead array. Results are representative of two to three independent experiments. Statistical testing by 2-way ANOVA with multiple comparisons. **, P<0.01; ****, P<0.0001.

FIG. 3 shows an example of clonal dynamics of γδ T cells upon pembrolizumab treatment. A, Diversity 50 index on TRG CDR3 clonotypes from sorted γδ T cells in peripheral blood. B, Frequency of top 10 clonotypes by CDR3 a.a sequence for E-013 (left) and E-035 (right). C, Frequency of top 10 clonotypes for E-020 (left), E-022 (middle), and E-034 (right). D, Frequency of top 10 TRD clonotypes of γδ T cells sorted from week 6 blood and from FFPE tumor. New lesion appeared in patient E-013 after they were taken off pembrolizumab due to intercurrent illness. E, Further frequency information for E-013 and E-035 at week 6.

FIG. 4 shows an example of Merkel cancer cell lines recognized by clonally expanded γδ T cells. A, Clonotypes were classified by CDR3 nucleotide sequence and colored according to absolute number of cells. B, γδTCR3-4 was highlighted as cells having CDR3 for TRG: CATWDGSRSYYKKLF (SEQ ID NO: 3), or TRD: CALGDYVLSDKLIF (SEQ ID NO: 6), or both. C, γδ TCRs were screened for reactivity against ovarian and Merkel cancer cell lines. Fold change in CD69 expression was normalized by dividing the Jurkat76 alone without any cell line or anti-CD3 stimulation. D, Example histogram of CD69 expression on J76 γδTCR3 after co-culture with MCC26 at 1:2 target to effector ratio. E, Jurkat76 cells were co-cultured with increasing number of target cancer cell lines. Non-transduced Jurkat76 cells are in light grey, mis-paired γδ TCRs are in white, γδTCR3-4 is in dark grey. Results are representative of two independent experiments.

FIG. 5 shows an example of γδ T cell clonotypes with distinct transcriptional profiles. A, UMAP plot of 3646 γδ T cells sorted from week 6 blood. (2541 and 1105 cells are from E-013 and E-035 respectively). B, Composition of each cluster by sample type. C, Composition of each cluster by clonotype size. D, γδTCR3-4 cells were subsetted and then reclustered with trajectory inference projection on top. E, Smoother plots of differentially expressed genes between the two lineages over pseudotime. F, Volcano plot of differentially expressed genes between γδ T cells from patients E-013 and E-035. DEG were determined by Wilcoxon rank-sum test with log 2 FC>0.4 and Bonferroni-adjusted P<0.01. G, Heatmap of top four differentially expressed genes between each cluster. H, Enriched gene signatures found between each cluster.

FIG. 6 shows an example of mispaired γδTCRs expressed on the cell surface. Cell surface expression of γδTCR and CD3 in transduced J76 cells is visible.

FIG. 7 shows an example of the extent of γδ TCR recognition of ovarian, breast, melanoma, and Merkel cancer cell lines. A, γδ TCRs were screened for reactivity against ovarian, breast and Merkel cancer cell lines. Fold change in CD69 expression was normalized by dividing the Jurkat76 alone without any cell line or anti-CD3 stimulation. B, example histogram of CD69 expression on J76 γδTCR3-4 after co-culture with MCC26 at 1:2 target to effector ratio. C,D Jurkat76 cells were co-cultured with increasing number of Merkel (C) or melanoma (D) target cancer cells.

FIG. 8 shows γδTCR3-4 recognizes MCC in B2M-independent manner. A, Expression of MHC class I family members in MCC26 cells that were CRISPRed with either scrambled or B2M-targeting guide RNAs. B, Co-culture of γδTCR3-4 J76 cells with MCC26 and increasing amounts of anti-CD1d antibody. C, Co-culture of γδTCR3-4 J76 cells with MCC26 and increasing amounts of anti-MR1 antibody. D, Co-culture of γδTCR3-4 J76 cells with MCC26 B2M knockout cells or with scrambled control.

FIG. 9 shows γδ TCR recognition of ovarian (SK-OV3), myeloma (RPMI-8226) and lung (A549) cancer cell lines. Transduced γδTCR-Jurkat76 cells were co-cultured with increasing number of SK-OV3 ovarian, RPMI-8226 myeloma, and A549 lung cancer cells.

### DETAILED DESCRIPTION

Provided herein is a novel γδ T cell receptor heterodimer. In some embodiments, there is provided a vector comprising a nucleic acid sequence encoding the novel γδ T cell receptor heterodimer or a T cell receptor γ peptide chain capable of forming a functional T cell receptor heterodimer together with a T cell receptor δ peptide chain, and/or a T cell receptor δ peptide chain capable of forming a functional T cell receptor heterodimer together with a T cell receptor γ peptide chain.

As used herein, a “protein heterodimer” is a macromolecular complex formed by two different proteins. In some embodiments, “protein heterodimer” may include a γ chain and a δ chain on a single polypeptide chain separated by a flexible linker.

In some embodiments, the T cell receptor heterodimer does not bind to cluster of differentiation 1 (CD1) CD1c and monomorphic antigen-presenting molecule MHC-related protein-1 (MR1) expressed on a tumour cell. In some embodiments, the T cell receptor does not bind to the γδ cell ligands CD1c, MR1 and, one or more, preferably all of, CD1d, MICA/B, MHC class I, MHC class II, EPCR, EpHA2, phosphoantigens, BTN3A1 and BTN3A2.

In some embodiments, the present invention is directed to a transduced T cell expressing a novel T cell receptor heterodimer comprising a first polypeptide comprising three γ CDRs comprising the sequences: EGSNGY (SEQ ID NO: 1) as CDR1γ, YDSYNSKVVLES (SEQ ID NO: 2) as CDR2γ and CATWDGSRSYYKKLF (SEQ ID NO: 3) as CDR3γ and a second polypeptide comprising three CDRs comprising the sequences: TSWWSYY (SEQ ID NO: 4) as CDR1δ, QGSDEQ (SEQ ID NO: 5) as CDR2δ, and CALGDYVLSDKLIF (SEQ ID NO: 6) as CDR3δ.

In some embodiments, the present invention is directed to a transduced T cell expressing a T cell receptor heterodimer comprising a first polypeptide comprising three γ CDRs and a second polypeptide comprising three δ CDRs, wherein the γ chain CDR 3 comprises CATWDGSRSYYKKLF (SEQ ID NO: 3). In some aspects, the δ chain CDR 3 comprises CALGDYVLSDKLIF (SEQ ID NO: 6).

The novel γδ T cell receptor heterodimer or a functional fragment thereof may be used in a bispecific antibody, that binds specifically to a protein expressed on the surface of a T cell, optionally an anti-CD3 antibody or fragment thereof that binds to a CD3 receptor of a T cell; and a T cell receptor heterodimer or a fragment thereof that binds to a tumor cell. The bispecific antibody can further include a linking region between the anti-CD3 antibody or fragment thereof and the T cell receptor heterodimer or fragment thereof, which can be an amino acid spacer. Methods of making bispecific antibodies are known to those of skill in the art, and may be found in the published literature e.g. Liddy, Nathaniel et al. “Monoclonal TCR-redirected tumor cell killing.” Nature medicine vol. 18, 6 (2012): 980-7; Bossi, Giovanna et al. “ImmTAC-redirected tumour cell killing induces and potentiates antigen cross-presentation by dendritic cells.” Cancer immunology, immunotherapy: CII vol. 63, 5 (2014): 437-48. The novel γδ T cell receptor heterodimer or a fragment thereof may be used in an antibody-drug conjugate comprising a T cell receptor heterodimer or a fragment thereof that binds to a tumor cell, conjugated to a drug. In some embodiments, the γδ T cell receptor is conjugated via a cleavable or non-cleavable linker to a cytotoxic drug. Various types of cytotoxic drugs are known to persons of skill in the art and include alkylating drugs, cytotoxic antibiotics, antimetabolite drugs, vinca alkaloids, photodynamic drugs and therapies, platinum drugs, taxanes and topisomerase inhibitors.

In some embodiments, the T cell receptor heterodimer or a fragment thereof may be the antigen-recognizing portion of a CAR-T construct. In another aspect, there is provided a modified T cell that expresses such a Chimeric Antigen Receptor (CAR).

The populations of T cells, bispecific antibodies and antibody-drug conjugates as provided herein can have application in the treatment of proliferative diseases and, in particular cancer, or an autoimmune disease in a subject.

The term “subject” as used herein includes all members of the animal kingdom including mammals, and, in particular, humans. In some embodiments, the subject has been diagnosed with a proliferative disease, in some embodiments cancer, or an autoimmune disease.

The term “cancer”, as used herein, may mean a malignant neoplasm that has undergone characteristic anaplasia with loss of differentiation, increased rate of growth, invasion of surrounding tissue, and is capable of metastasis. Metastatic cancer is a cancer at one or more sites in the body other than the site of origin of the original (primary) cancer from which the metastatic cancer is derived.

The term “tumor”, as used herein, refers to a neoplasm or an abnormal mass of tissue that is not inflammatory, which arises from cells of pre-existent tissue. A tumor can be either benign (noncancerous) or malignant (cancerous). Tumors can be solid or hematological. Examples of solid tumors include sarcomas and carcinomas. Examples of hematological tumors include, but are not limited to: leukemias, lymphoma, myelomas.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment.

In some embodiments, populations of T cells as provided herein may be used in a T cell therapy in a subject, optionally an allogeneic cell therapy. Advantageously, populations of T cells as provided herein may be suitable for use in an allogeneic T cell therapy that does not require human leukocyte antigen (HLA) matching.

In some embodiments, the invention includes methods of treating a tumor having downregulated MHC class I antigen presentation or a cancer that is associated with downregulation of MHC class I antigen presentation. The loss of MHC I antigen presentation makes cancers less visible to the immune system and, in particular, targeting by CD8+ T cells. Loss of MHC I expression has been associated with resistance to checkpoint blockade and adoptive immunotherapy. Methods of identifying a tumor having downregulated MHC class I antigen presentation are known to those of skill in the art and include immunohistochemistry analysis of 2-microglobulin (B2M) and MHC class I expression levels and DNA sequencing of B2M mutations or loss of Human leukocyte antigen (HLA) heterozygosity as determined by analysis in excised or biopsied tumors; e.g. in some embodiments, MHC class I antigen presentation may be downregulated in >90% of the tumor cells.

The Example provides evidence in particular that the cell populations, bispecific antibodies and antibody-drug conjugates described herein can have utility in the treatment of Merkel cell cancer, melanoma and breast cancer.

As detailed in the Example, the present inventors provide evidence that in some cancer patients, γδ T cells exhibit an activated phenotype, and anti-PD-1 can enhance γδ T cell proliferation, as well as the production of IFNγ and IL-2.

In some embodiments, the subject of treatment has previously been subject to an immunotherapy treatment regimen. The term “immunotherapy” as used herein refers to methods and compositions that induce or enhance the targeting or destruction of cancer cells by the immune system. Immunotherapies include immune checkpoint inhibitors, monoclonal antibodies, T cell therapy (e.g. CAR-T), oncolytic virus therapy, and cancer vaccines. In some embodiments, the subject of treatment has previously been administered an immune checkpoint inhibitor, optionally a PD-1 inhibitor. Known immune checkpoint inhibitors include, but are not limited to pembrolizumab, ipilimumab, nivolumab, atezolizumab, avelumab, and durvalumab.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

The advantages of the present invention are further illustrated by the following examples. The examples and their particular details set forth herein are presented for illustration only and should not be construed as a limitation on the claims of the present invention

### Example

Expansion of γδ T Cells in Blood and Tumor after Pembrolizumab Treatment

Six Merkel cell carcinoma (MCC) patients were studied from a trial of 106 patients recruited and enrolled in one of five cohorts: head and neck squamous cell carcinoma, triple-negative breast cancer, high-grade serous carcinoma, metastatic melanoma and mixed solid tumors.²²⁻²⁴ All patients were naïve to anti-PD-1/PD-L1 therapy prior to enrollment, and were treated with 200 mg of pembrolizumab given every three weeks intravenously, for a maximum of up to two years. Tumor lesions were biopsied at baseline (within 28 days of study treatment) and on-treatment (week six or week nine). Pooled core biopsies or tissue samples were minced into 2-4 mm³ fragments and then mechanically and enzymatically digested with the gentle MACS dissociator (Miltenyi Biotec, Catalog #130-093-235) and the human tumor dissociation kit (Miltenyi, Catalog #130-095-929). Peripheral blood samples were collected at baseline, week 3, week 6, week 9, week 15 and every nine weeks thereafter and at the end-of-treatment.

Flow cytometry analyses were performed to characterize different T cell phenotypes. Cells were incubated with Fc receptor inhibitor (Thermo Fisher Scientific, Catalog #14-9161-73) and eFluor506 fixable viability (Thermo Fisher Scientific, Catalog #65-0866-14), followed by primary antibody cocktails, and then fixed with 4% paraformaldehyde. Primary antibody stain and fixation was performed for 30 minutes at 4° C. and protected from light. For biotinylated antibodies, a 15-minute incubation with streptavidin-conjugated fluorochrome was added after antibody staining. For intracellular staining, cells were fixed, permeabilized and stained with the FoxP3 Transcription Factor set (Thermo, Catalog #00-5523-00). Flow cytometry data was then acquired on a LSR Fortessa and analyzed using FlowJo v10 (BD Biosciences).

For fluorescent-activated cell sorting (FACS), cells were incubated with Fc receptor inhibitor and eFluor506 fixable viability, and then stained with anti-CD3-PE-Cy7, anti-γδ TCR-PE, anti-CD8-FITC, anti-CD4-APC, and sorted with FACS Aria Fusion (BD Biosciences).

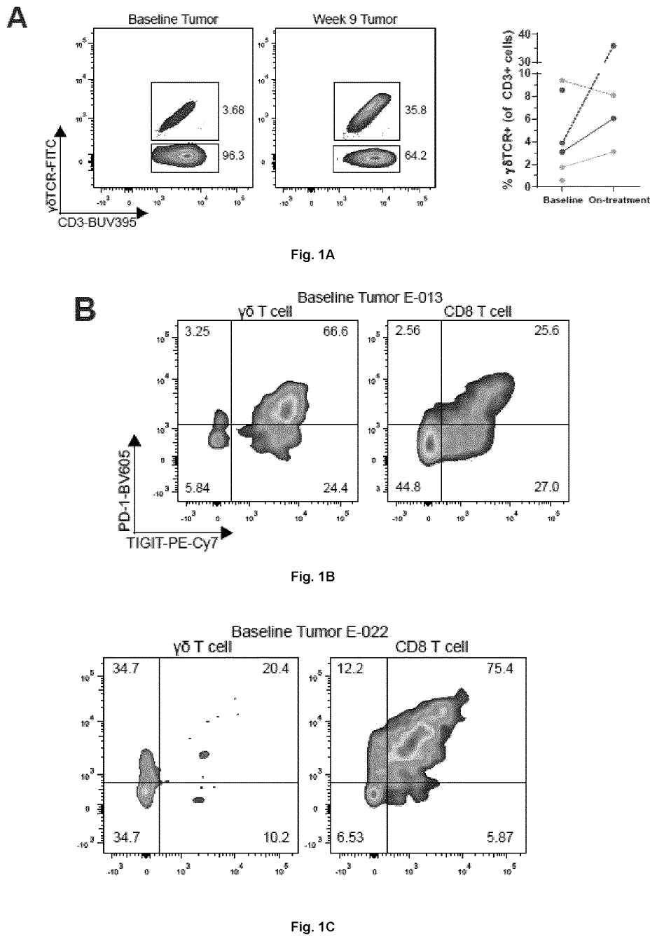

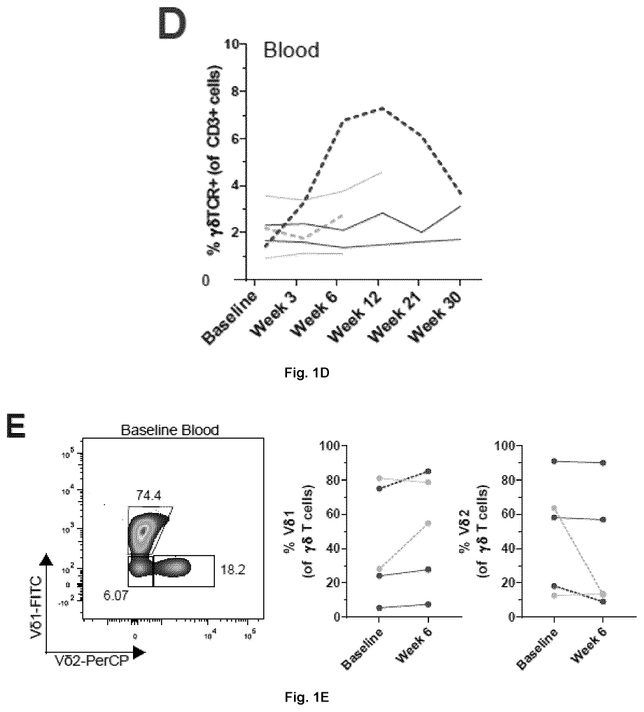

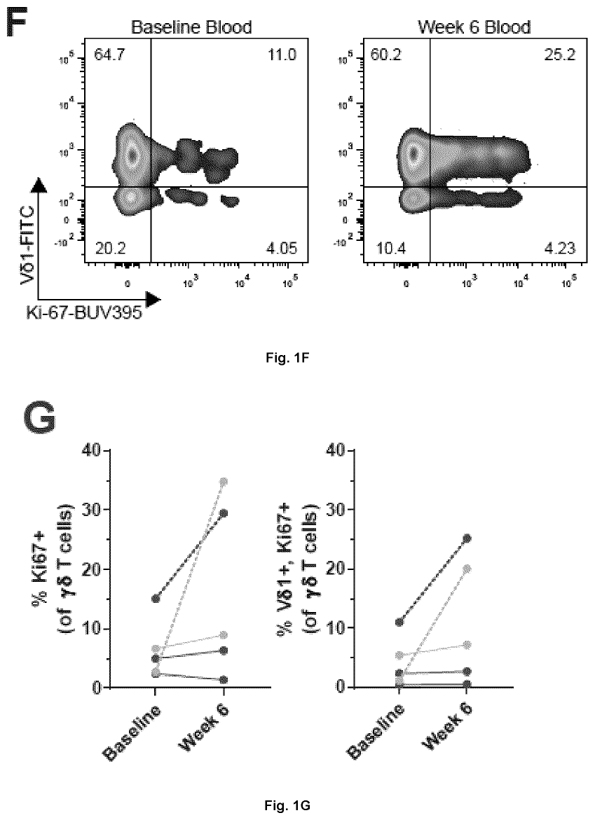

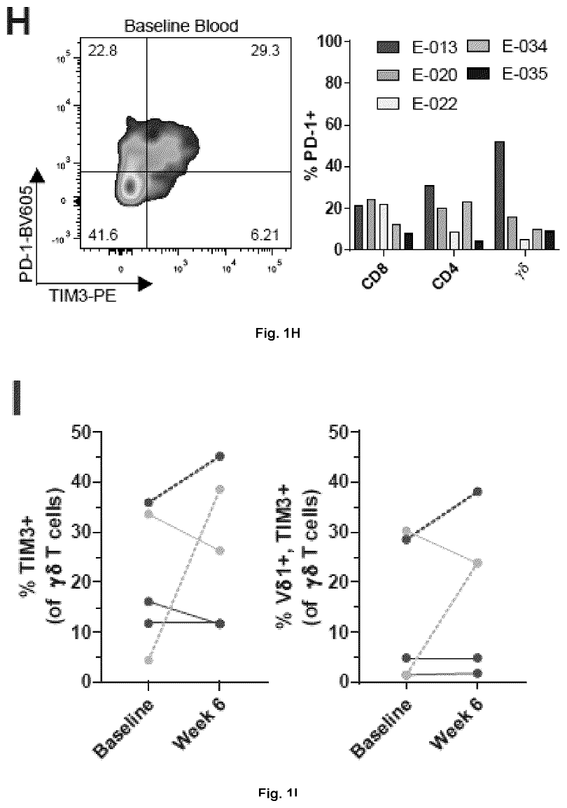

For 3 of 4 MCC patients with on-treatment data, γδ T cells expansion ranged from 1.8-fold-9.7-fold, with patient E-013 having an almost 10-fold expansion (3.68 to 35.8%) in the frequency of γδ T cells. Patient E-013 experienced a complete response to therapy (FIG. 1A) and at baseline their γδ T cells had the highest expression of PD-1 and TIGIT as compared to their conventional CD8 and CD4 T cells (FIG. 1B). This was not observed for patients that did not show robust expansion of γδ T cells and did not have high expression of PD-1 or TIGIT on γδ T cells (FIG. 1C).

Analysis of the peripheral blood from patient E-013 showed a four-fold expansion of γδ T cells (1.4 to 7.28%) that peaked at week 12 and then subsequently declined (FIG. 1D). There was a wide range (8.98-91.1%) of Vδ2 cells observed amongst the MCC patients (FIG. 1E). Patients E-013 and E-035 also had an increase in Vδ1 cells that coincided with a decrease in Vδ2 cells after pembrolizumab treatment (FIG. 1E). Both E-013 and E-035 had an increase in Ki-67+γδ T cells at week 6 of anti-PD-1 treatment, suggesting the change in frequency of γδ T cell subsets was due to proliferation (FIG. 1F and G). Furthermore, this proliferation was primarily attributed to the Vδ1 subset (FIG. 1F and G). γδ T cells from patients E-013 and E-035 had higher expression of PD-1 in peripheral blood compared to their CD8 or CD4 T cells (FIG. 1H). Additionally, the Vδ1 subset had increased expression of TIM-3 upon pembrolizumab treatment (FIG. 11).

### In Vitro PD-1 Blockade

To investigate whether anti-PD-1 treatment could directly enhance the effector functions of γδ T cells, γδ T cells from healthy donor PBMCs were enriched with TCRγ/δ+ T Cell Isolation Kit (Miltenyi, Catalog #130-092-892) and then labelled with 1 μM eF450 proliferation dye (Thermo, Catalog #5-0842-85). MS-1 cells were pulsed with 10 μM (E)-4-Hydroxy-3-methyl-but-2-enyl pyrophosphate (HMB-PP) (Sigma, Catalog #95098) or a vehicle control for 4 hours at 37° C. 50,000 γδ T cells were then co-cultured with 5000 MS-1 cells for a 10:1 effector to target ratio in the presence of 100 IU/mL IL-2 (Chiron) and either 20 μg/mL human IgG4 isotype or pembrolizumab (Merck). After three days, proliferation was measured by flow cytometry and supernatant was collected for cytokine measurement using the LEGENDplex Th1 panel (BioLegend, Catalog #741035).

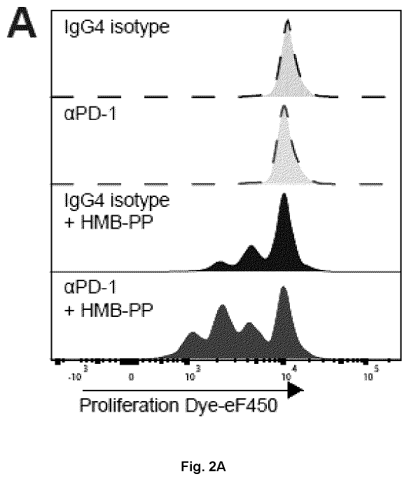

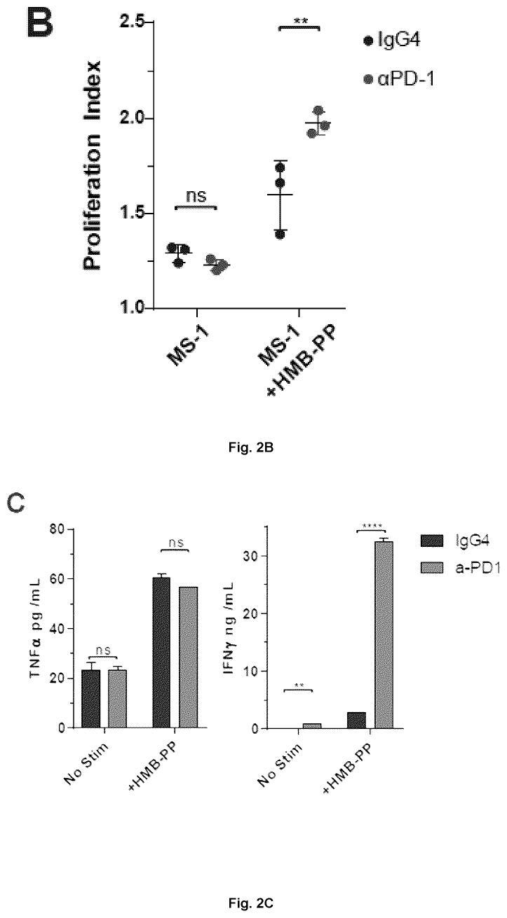

In the absence of the phosphoantigen, the MS-1 cells did not induce γδ T cell proliferation, even with the addition of αPD-1 (FIG. 2A). When MS-1 cells were pulsed with HMB-PP, the γδ T cells were able to proliferate, and this proliferation was further enhanced with anti-PD-1 antibody (FIG. 2A and B). When cytokine levels were measured in the supernatant, there was a substantial increase in IFNγ production, but not TNFα (FIG. 2C). IL-2 production was measured by intracellular cytokine staining. It was found that αPD-1 treatment could increase IL-2 production in both the unstimulated and HMB-PP pulsed conditions (FIG. 2D). These experiments demonstrate that anti-PD-1 can directly enhance in vitro γδ T cell proliferation, as well as the production of IFNγ and IL-2.

### Detection of Peripheral Blood γδ T Cells in the Tumor

### Clonal Expansion of γδ T Cells

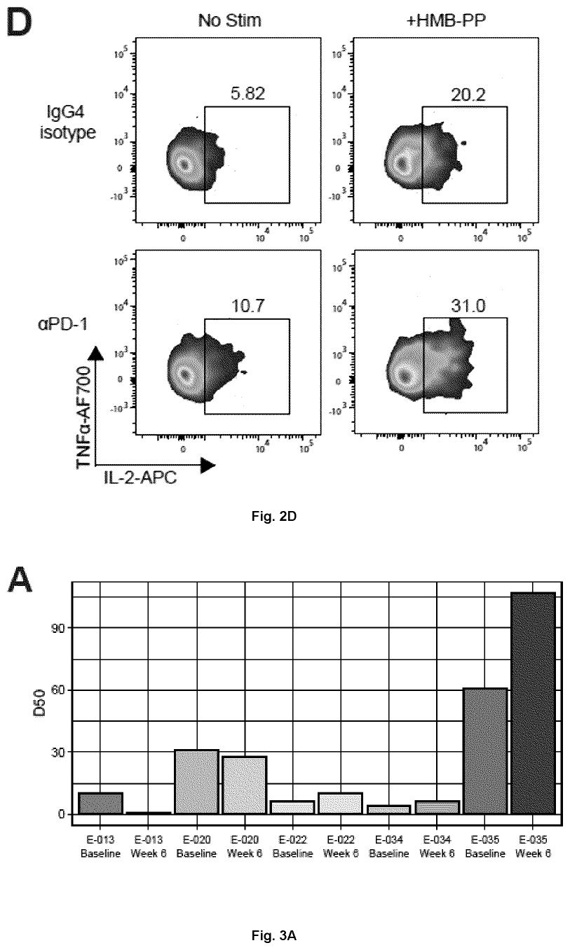

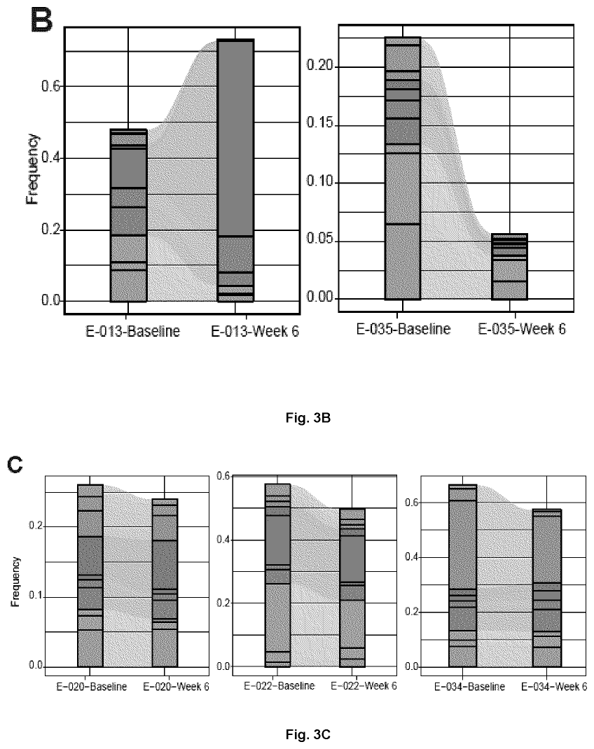

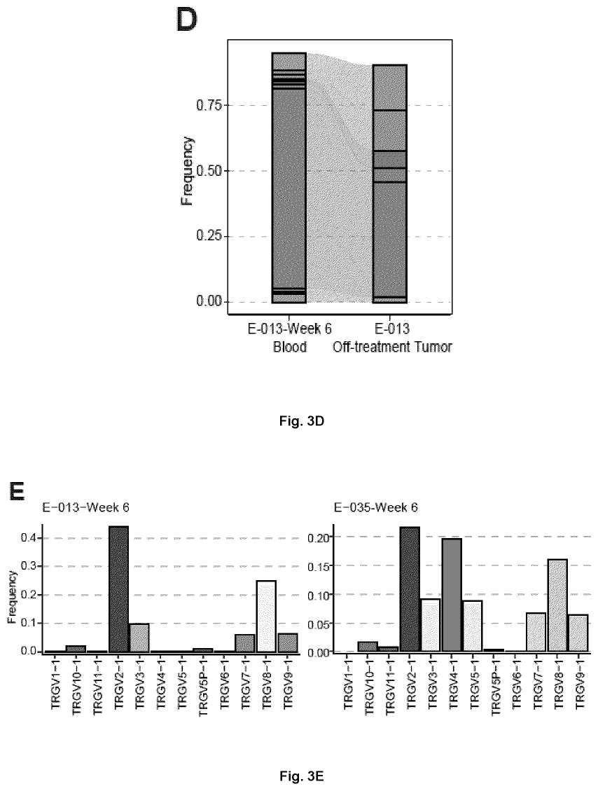

γδ T cells were collected from peripheral blood at baseline and week 6. TRG sequencing was performed to explore the diversity and clonal dynamics upon pembrolizumab treatment. Using D50 index as a measurement of diversity (minimum number of clonotypes that comprise 50% of the total repertoire), patient E-013 had a decrease in diversity (from 10 to 1 clonotypes), while E-035 had an increase in diversity (from 61 to 107 clonotypes) upon PD-1 blockade (FIG. 3A). Considering the frequency of the top 10 clonotypes by CDR3 amino acid sequence, one clonotype in patient E-013 was found to increase from 10.8 to 54.6% (FIG. 3B), while in patient E-035, the frequencies of the top 10 clonotypes shrank to comprise only 5.6% of the total repertoire (FIG. 3B). Patients E-020, E-022, and E-034 had minimal changes to their TRG repertoire (FIG. 3C).

### Detection of Expanded Clonotypes in the Tumor

γδ T cells from week 6 PBMCs were purified and compared the CDR3 sequences with genomic DNA extracted from formalin-fixed and paraffin embedded (FFPE) tissue of a surgically resected lesion after the patient was taken off pembrolizumab after 34 cycles due to intercurrent illness. TRD sequencing was used to compare the TCR repertoire between γδ T cells in peripheral blood and FFPE tissue. When observing the top 10 clonotypes, the TRD repertoire was highly comparable between blood and tumor, with the top clone representing 76.3% in week 6 blood and 43.9% in tumor (FIG. 3D).

### Bulk TCR Sequencing and Jurkat76 Transduction

Genomic DNA from sorted γδ T cells was extracted with AllPrep DNA/RNA (Qiagen, Catalog #80284). For FFPE tissue, DNA was extracted using AllPrep DNA/RNA FFPE (Qiagen, Catalog #80234). DNA was sent for TRG or TRA/D sequencing by Adaptive Biotechnologies. Reads were aligned and filtered for in-frame CDR3 sequences by Adaptive Biotechnologies. Downstream analysis was performed with immunarch package. Jurkat 76 cells were transduced with individual TCRg and TCRd genes. Briefly, full length TRG or TRD chains were synthesized (Invitrogen GeneArt Gene Synthesis) from partial CDR3 variable region reads and cloned into pMX expression vector for retroviral packaging. Individual TRG and TRD genes were transduced into Jurkat76 using spinfection method and after 5 days, TCR transfectants were selected to purity (>95% purity) using CD3 Microbeads (Miltenyi Biotec).

### Single Cell RNA and TCR Sequencing

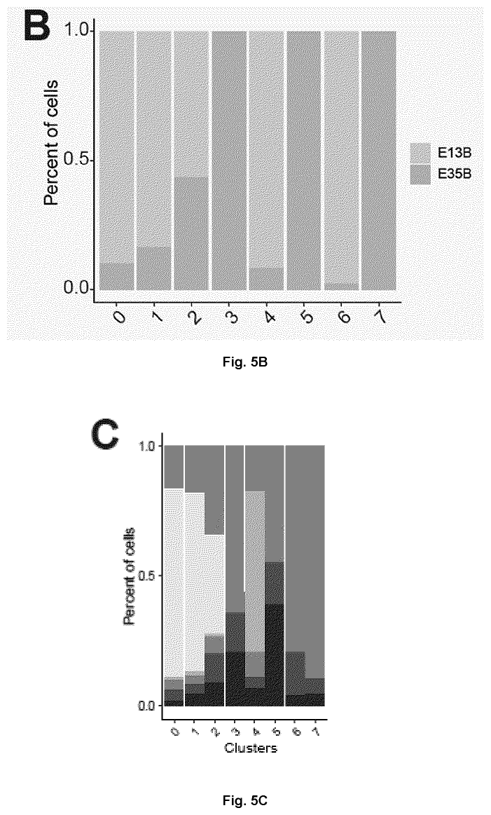

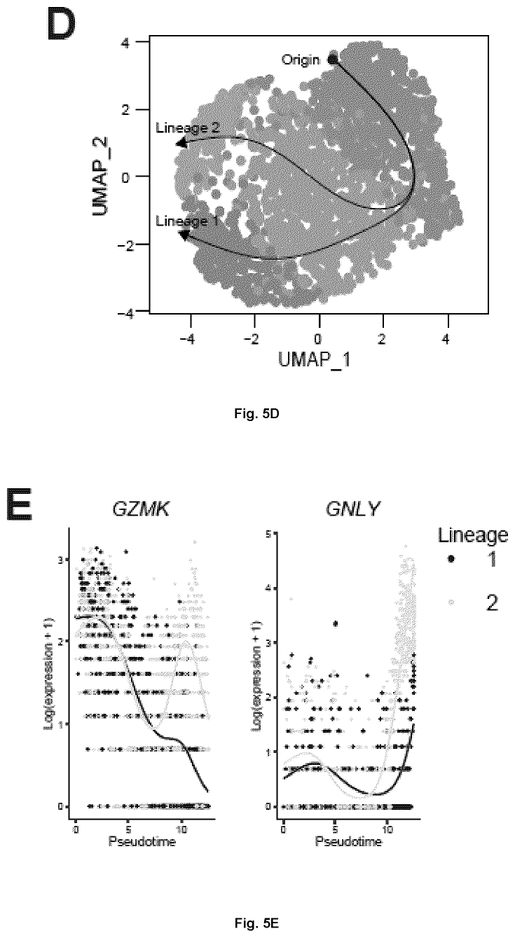

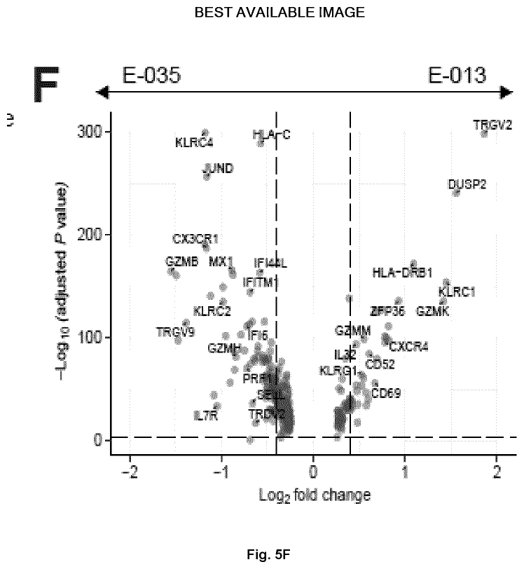

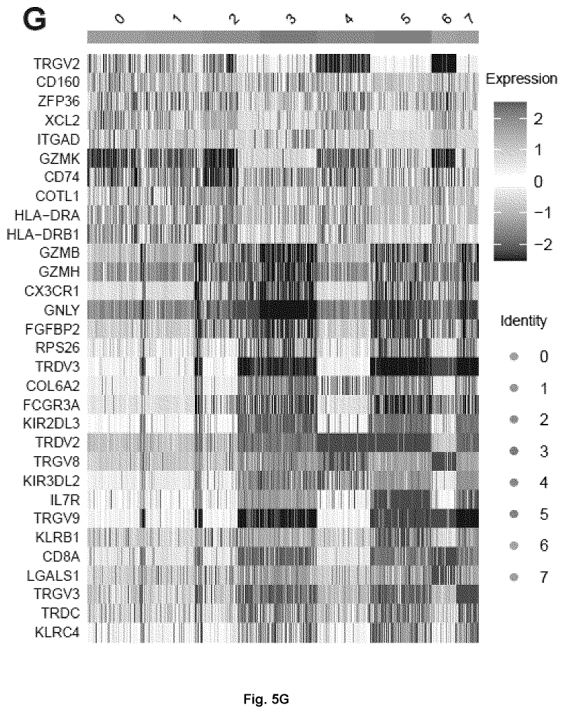

γδ T cells from week 6 peripheral blood of patients E-013 and E-035 were sorted and 3,646 cells were obtained across eight clusters (FIG. 5). Single cell libraries were created with 10× Genomics Chromium 5′ Kit v2 by the Princess Margaret Genomics Centre. γδ TCR libraries were prepared according to a published protocol.²⁵ Analysis of single cell data was performed using Seurat²⁶, scRepertoire²⁷ and escape.²⁸

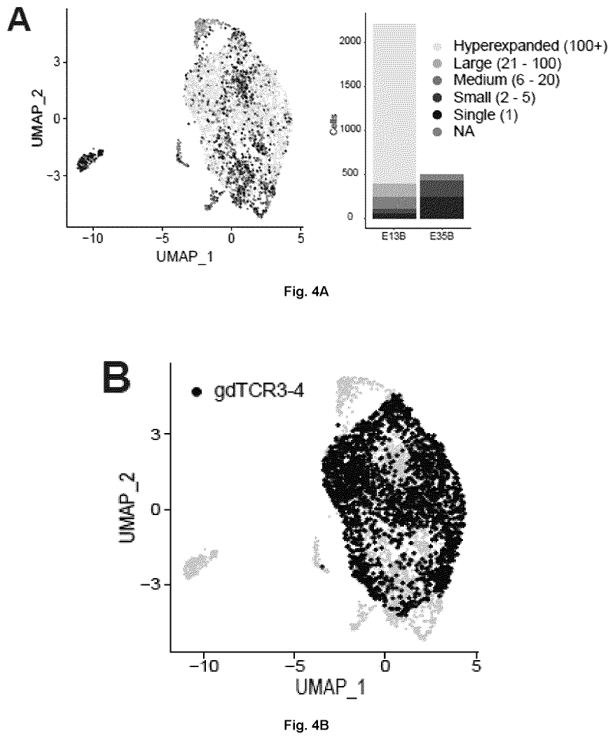

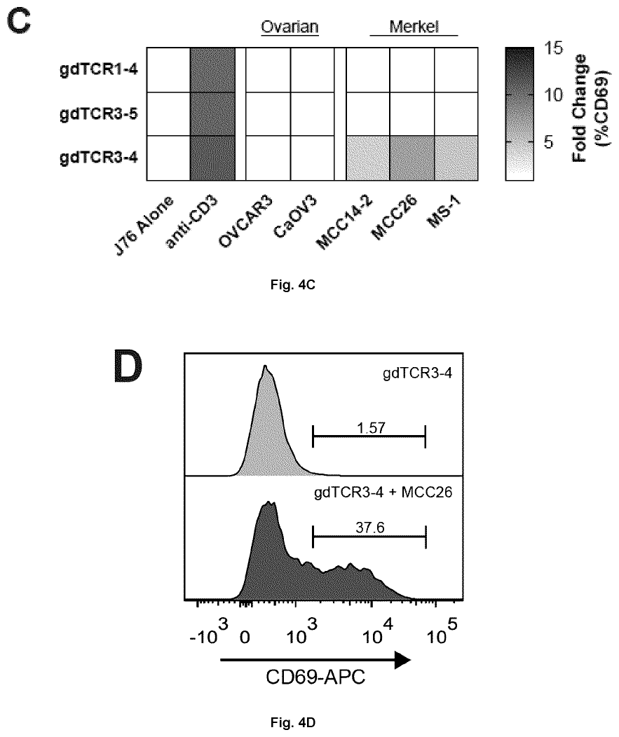

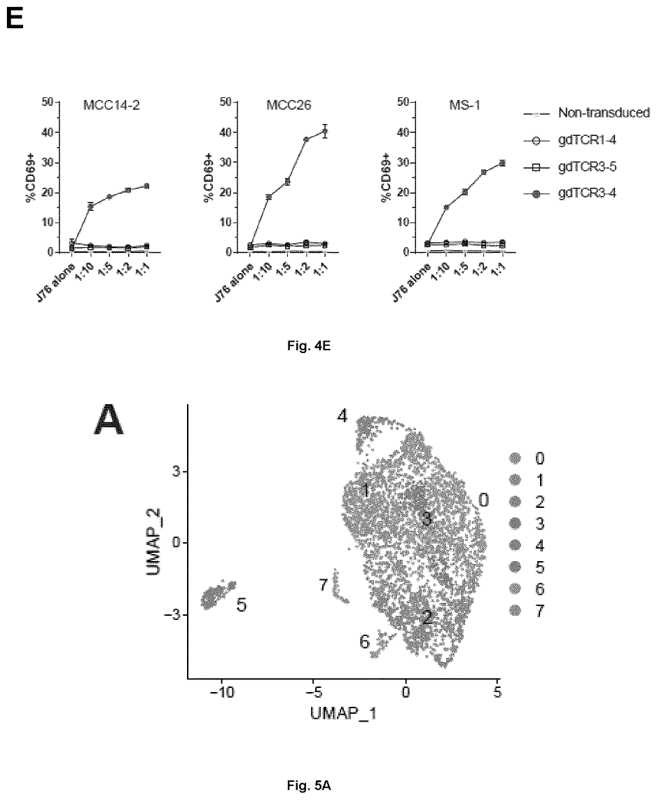

From the TCR sequencing, hyperexpanded and clonal γδ T cells were identified (FIG. 4A) that were predominantly found in clusters 0, 1 and 2 (FIG. 5). Considering the most differentially expressed genes between each cluster, cluster 0 were TRGV2+ cells that expressed ZFP36 (FIG. 5). Cluster 1 cells had high expression of genes associated with MHC class II antigen presentation such as CD74 invariant chain, HLA-DRA and HLR-DRB1, while cytotoxic cells expressing GZMB, GZMH and GNLY were found in cluster 2 (FIG. 5). There was one particular clonotype that was identical to the top TRG and TRD CDR3 amino acid sequences found in the bulk TCR sequencing data (FIG. 3B and D) which was designated as clonotype γδTCR3-4 (FIG. 4B). The γδTCR3-4 clonotype totaled 1,913 cells, while clonotypes from E-035 never surpassed 20 cells. These cells were subsetted out and Slingshot was used to perform trajectory inference analysis. It was found that the γδTCR3-4 clonotype branched into two distinct lineages, and across pseudotime there was higher expression of GZMK and GNLY in Lineage 2 (FIG. 5D and E). Considering differentially expressed genes between patients E-013 and E-035, there was higher expression of interferon-stimulated genes MX1, IFI6, IFI44L and IFITM in the γδ T cells from patient E-035 (FIG. 5F).

Molecular Signatures Database was used to perform gene set enrichment analysis. Clusters 0, 1, 4 and 6 grouped closely together and the majority of their cells came from patient E-013. These clusters were enriched for T cell response to tumor, T cell mediated cytotoxicity and γδ T cell activation (FIG. 5). By contrast, clusters 3, 5 and 7 had all their cells originating from patient E-035 and were associated with cytokine production (FIG. 5). Signatures associated with IFNγ production, PD-1 ligation and PD-1 high-expressing cells were dispersed among clusters from both patients. These findings indicate that γδTCR3-4 cells from patient E-013 were clonally expanded and have cytotoxic potential.

Co-Culture of Jurkat76.gdTCR and Cancer Cell Lines

To determine if γδTCR3-4 could recognize Merkel cancer cells, γδTCR3-4 was cloned and expressed into Jurkat76 cells that lacked the endogenous TCR and then co-cultured with various cell lines from Merkel, melanoma, breast and ovarian cancers. Additionally, using the next frequent clonotype, either the γ chain (γδTCR3-5) or the δ chain (γδTCR1-4) were mis-paired to generate negative control cells.

The following Merkel cell carcinoma lines were used: MCC14/2 (Sigma-Aldrich Catalog #10092303), MCC26 (Sigma-Aldrich Catalog #10092304), MS-1 (Sigma-Aldrich Catalog #09111802). The ovarian cancer cell lines Caov-3 (American Type Culture Collection, Catalog #HTB-75) and OVCAR3 (laboratory of Dr. T. Mak, Princess Margaret Cancer Centre) were used. Finally, the breast cancer cell lines HCC1143 and T47D (laboratory of Dr. M. Reedijk, Princess Margaret Cancer Centre) and the melanoma cell lines A375, 624-mel and 888-mel were used (laboratory of Dr. S. Rosenberg, National Cancer Institute).

The Merkel cell carcinoma lines were cultured in RPMI-1640 (Gibco) and supplemented with 20% fetal calf serum (FCS), L-glutamine, and HEPES. The Caov-3 cell line was cultured in DMEM and supplemented with 10% FCS, and L-glutamine. OVCAR3 cells were cultured in RPMI-1640 and supplemented with 10% FCS, 0.01 mg/ml bovine insulin, and L-glutamine. Jurkat 76 cells (laboratory of Dr. M. Heemskerk, Leiden University Medical Center) were cultured in RPMI-1640 and supplemented with 10% FCS, and L-glutamine. All cell lines were maintained in presence of penicillin, streptomycin, and gentamicin.

The breast cancer cell line T47D was cultured in RPMI-1640 and supplemented with 10% FCS, 0.01 mg/ml bovine insulin, and L-glutamine and HCC1143 was cultured in RPMI-1640 and supplemented with 10% FCS, and L-glutamine. The melanoma cancer cell lines A375, 624-mel and 888-mel were cultured in RPMI-1640 and supplemented with 10% FCS and L-glutamine.

Cells were trypsinized, washed and then 25,000 cells were seeded on a 96 well plate and incubated overnight. Positive control wells were pre-coated with 5 μg/mL of anti-CD3 OKT3 (Biolegend, Catalog #317326) overnight at 37° C. and then washed. Afterwards, 50,000 Jurkat76 cells were added. Following a 16-20-hour incubation, cells were stained with viability dye, anti-CD3-PE (BD Biosciences, Catalog #566683) and anti-CD69-APC (Biolegend, Catalog #310910). For calculating fold change of % CD69, Jurkat76 cells were gated by CD3 expression and then the frequency of CD69 was normalized by

$$\frac{\% CD 69 of Condition}{\% CD 69 of Jurkat 76 alone} .$$

For dose-dependent TCR recognition of cancer cell lines, 5000, 10,000, 25,000 and 50,000 cancer cells were co-cultured with 50,000 Jurkat76 cells.

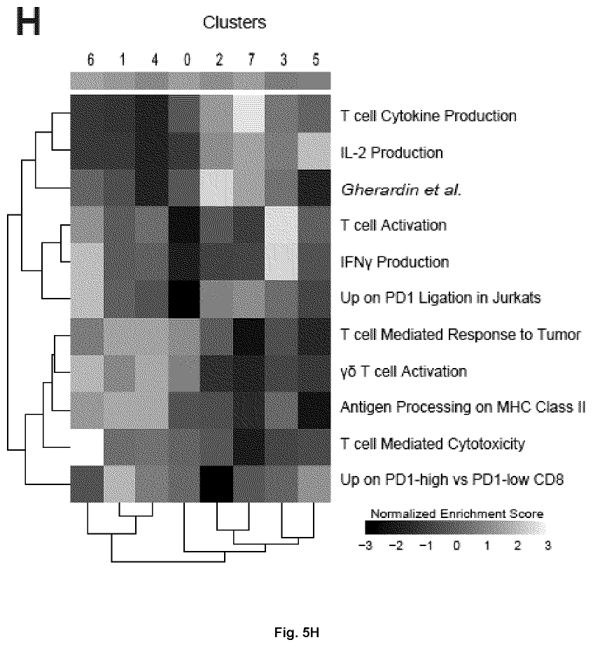

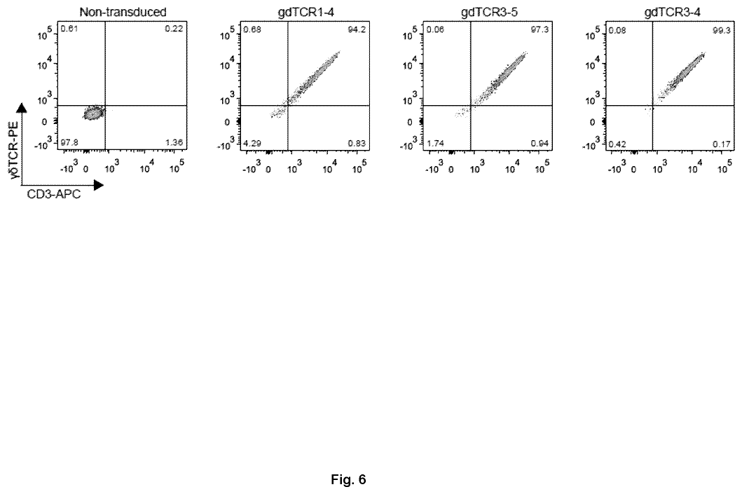

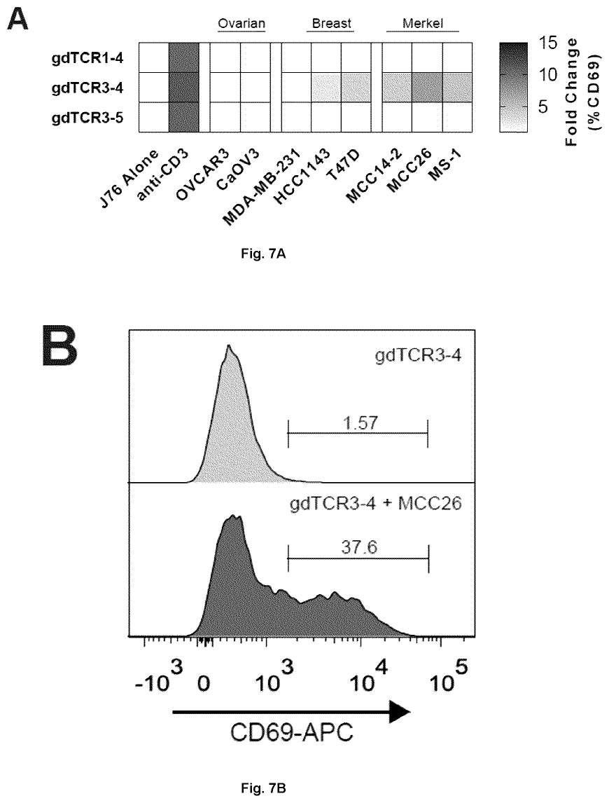

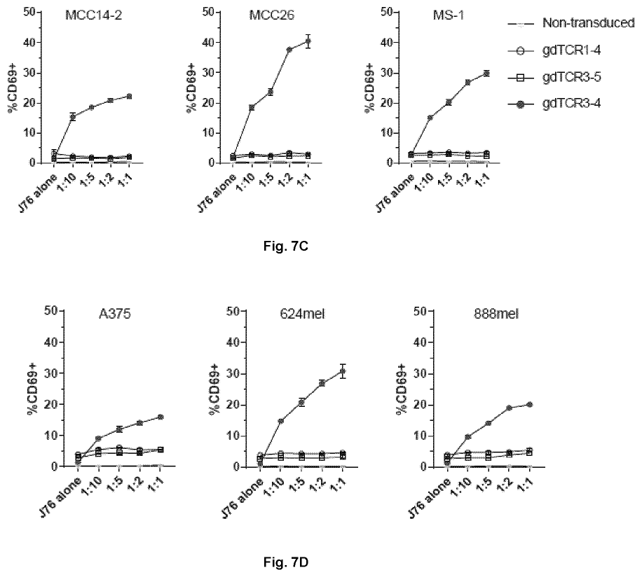

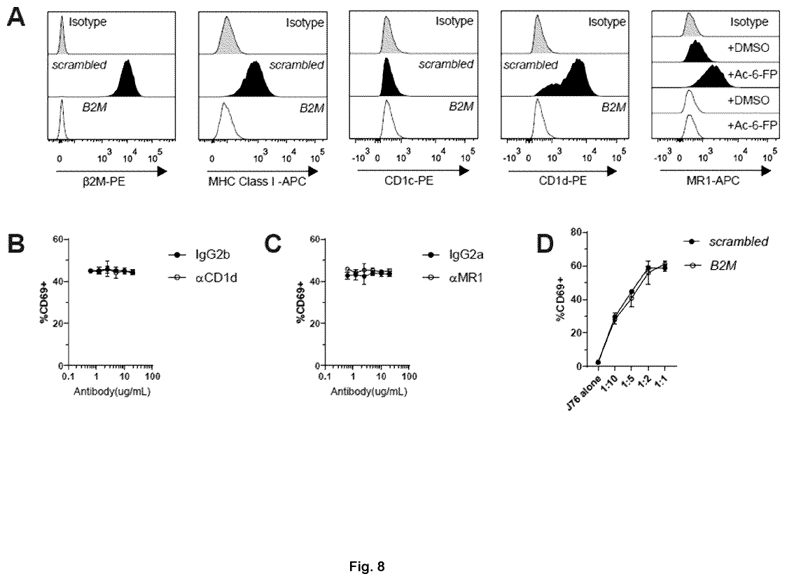

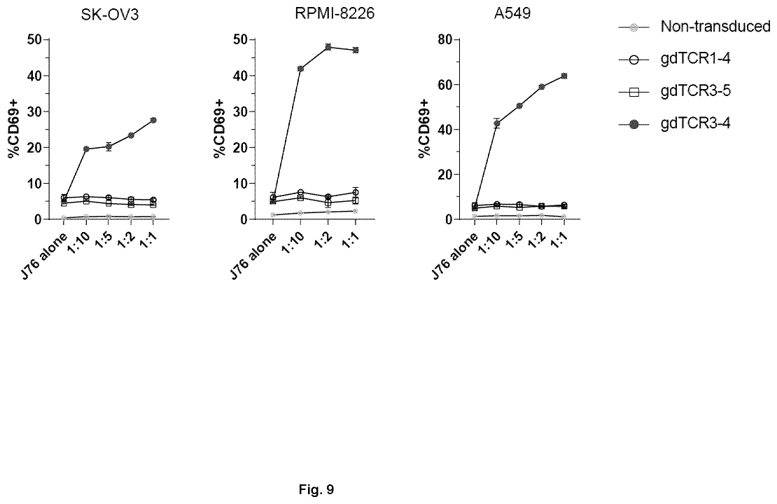

All three γδ TCR were stably expressed and maintained on Jurkat76 cells (FIG. 6, Table 1). Using CD69 upregulation as a readout of TCR reactivity, all γδTCR Jurkat76 cells showed responses to anti-CD3 stimulation (FIG. 7A). Only γδTCR3-4 were able to recognize the Merkel cancer cell lines MCC14/2, MCC26 and MS-1, but not the ovarian cell lines OVCAR3 and CaOV3 (FIG. 4C and D). gdTCR3-4 further showed reactivity towards breast cancer (HCC1143 and T47D) and melanoma (A375, 624-mel and 888-mel). Co-cultures with increasing number of target cancer cells were performed to demonstrate recognition of Merkel carcinoma cell lines. Amongst all the Merkel cancer cell lines, only γδTCR3-4 but not the mis-paired γδ TCRs, nor the non-transduced Jurkat76 parental line had a dose-dependent increase in CD69 expression (FIG. 4E, FIG. 7C).

TABLE 1
CDR3 amino acid sequences of
γσTCRs cloned into J76 Jurkat cells

| | TRGV | CDR3 γ | TRGJ | TRDV | CDR3 σ | TRDJ |
|---|---|---|---|---|---|---|
| gdTCR1-4 | 2 | CATWDGSRSYYKKLF | 1 | 1 | CALDLGGRGPPPDKLIF | 1 |
| | | (SEQ ID NO: 3) | | | (SEQ ID NO; 8) | |
| gdTCR3-5 | 8 | CATWDPYYKKLF | 1 | 1 | CALGDYVLSDKLIF | 1 |
| | | (SEQ ID NO: 7) | | | (SEQ ID NO; 6) | |
| gdTCR3-4 | 2 | CATWDGSRSYYKKLF | 1 | 1 | CALGDYVLSDKLIF | 1 |
| | | (SEQ ID NO: 3) | | | (SEQ ID NO: 6) | |

TABLE 2
γσTCR3-4 CDR amino acid sequences

| chain | cdr1 | cdr2 | cdr3 |
|---|---|---|---|
| TRG | EGSNGY | YDSYNSK | CATWDGSR |
| | (SEQ ID | VVLES | SYYKKLF |
| | NO: 1) | (SEQ ID | (SEQ ID |
| | | NO: 2) | NO: 3) |
| TRD | TSWWSYY | QGSDEQ | CALGDYVL |
| | (SEQ ID | (SEQ ID | SDKLIF |
| | NO: 4) | NO: 5) | (SEQ D |
| | | | NO: 6) |

TABLE 3
γσTCR3-4 CDR encoding sequences

| chain | cdr1_nt | cdr2_nt | cdr3_nt |
|---|---|---|---|
| TRG | GAAGGAA | TATGACT | TGTGCCA |
| | GTAACGG | CCTACAA | CCTGGGA |
| | CTAC | CTCCAAG | CGGGTCC |
| | (SEQ ID | GTTGTGT | CGAAGCT |
| | NO: 9) | TGGAATC | ATTATAA |
| | | A | GAAACTC |
| | | (SEQ ID | TTT |
| | | NO: 10) | (SEQ ID |
| | | | NO: 11) |
| TRD | ACAAGTT | CAGGGTT | TGTGCTC |
| | GGTGGTC | CTGATGA | TTGGGGA |
| | ATATTAT | ACAG | CTACGTT |
| | (SEQ ID | (SEQ ID | CTATCCG |
| | NO: 12) | NO: 13) | ATAAACT |
| | | | CATCTTT |
| | | | (SEQ ID |
| | | | NO: 14) |

Co-Culture of Jurkat76.gdTCR with Further Cancer Cell Lines

To determine if the γδTCR3-4 could recognize further cancer cells, γδTCR3-4 was cloned and expressed into Jurkat76 cells that lacked the endogenous TCR and co-cultured them with additional cancer cell lines per the methodology described in the preceding section. Again as controls, the next most frequent clonotype was used to generate mis-paired γδTCR; either the γ chain (γδTCR3-5) or the δ chain (γδTCR1-4). Using CD69 upregulation as a readout of TCR reactivity, the γδTCR3-4 could recognize SK-OV3 ovarian cancer, RPMI-8226 myeloma, and A549 lung carcinoma, evidencing that the stimulating ligand can be shared between other cancer types. As γδ TCRs are not HLA-restricted, and γδTCR3-4 can recognize different cancer histologies.

As can be seen therefore, the examples described above and illustrated are intended to be exemplary only. The scope is indicated by the appended claims.

### REFERENCES

1. D'Angelo, S. P. et al. Avelumab in patients with previously treated metastatic Merkel cell carcinoma: Long-term data and biomarker analyses from the single-arm phase 2 JAVELIN Merkel 200 trial. J Immunother Cancer 8, (2020).
2. Nghiem, P. T. et al. PD-1 Blockade with Pembrolizumab in Advanced Merkel-Cell Carcinoma. New England Journal of Medicine 374, 2542-2552 (2016).
3. Kaufman, H. L. et al. Avelumab in patients with chemotherapy-refractory metastatic Merkel cell carcinoma: a multicentre, single-group, open-label, phase 2 trial. Lancet Oncol 17, 1374-1385 (2016).
4. Im, S. J. et al. Defining CD8+ T cells that provide the proliferative burst after PD-1 therapy. Nature 537, 417-421 (2016).
5. Utzschneider, D. T. et al. T Cell Factor 1-Expressing Memory-like CD8+ T Cells Sustain the Immune Response to Chronic Viral Infections. Immunity 45, 415-427 (2016).
6. Tanaka, Y. et al. Natural and synthetic non-peptide antigens recognized by human gamma delta T cells. Nature 375, 155-8 (1995).
7. Vavassori, S. et al. Butyrophilin 3A1 binds phosphorylated antigens and stimulates human γδ T cells. Nat Immunol 14, 908-916 (2013).
8. le Nours, J. et al. A class of γδ T cell receptors recognize the underside of the antigen-presenting molecule MR1. Science (1979) 366, 1522-1527 (2019).
9. Wun, K. S. et al. T cell autoreactivity directed toward CD1c itself rather than toward carried self lipids. Nat Immunol 19, 397-406 (2018).
10. Groh, V., Steinle, A., Bauer, S. & Spies, T. Recognition of stress-induced MHC molecules by intestinal epithelial gammadelta T cells. Science (1979) 279, 1737-40 (1998).
11. Benveniste, P. M. et al. Generation and molecular recognition of melanoma-associated antigen-specific human γδ T cells. Sci Immunol 3, 4036 (2018).
12. Mueller, S. N. & Ahmed, R. High antigen levels are the cause of T cell exhaustion during chronic viral infection. Proc Natl Acad Sci USA 106, 8623-8 (2009).
13. Barber, D. L. et al. Restoring function in exhausted CD8 T cells during chronic viral infection. Nature 439, 682-687 (2006).
14. Baitsch, L. et al. Exhaustion of tumor-specific CD8+ T cells in metastases from melanoma patients. Journal of Clinical Investigation 121, 2350-2360 (2011).
15. Barber, D. L. et al. Restoring function in exhausted CD8 T cells during chronic viral infection. Nature 439, 682-687 (2006).
16. Jin, H. T. et al. Cooperation of Tim-3 and PD-1 in CD8 T-cell exhaustion during chronic viral infection. Proc Natl Acad Sci USA 107, 14733-14738 (2010).
17. Alfei, F. et al. TOX reinforces the phenotype and longevity of exhausted T cells in chronic viral infection. Nature 571, 265-269 (2019).
18. Scott, A. C. et al. TOX is a critical regulator of tumour-specific T cell differentiation. Nature 571, 270-274 (2019).
19. Khan, O. et al. TOX transcriptionally and epigenetically programs CD8+ T cell exhaustion. Nature 571, 211-218 (2019).
20. Pauken, K. E. et al. Epigenetic stability of exhausted T cells limits durability of reinvigoration by PD-1 blockade. Science (1979) 354, 1160-1165 (2016).
21. Sen, D. R. et al. The epigenetic landscape of T cell exhaustion. Science 354, 1165-1169 (2016).
22. Clouthier, D. L. et al. An interim report on the investigator-initiated phase 2 study of pembrolizumab immunological response evaluation (INSPIRE). J Immunother Cancer 7, (2019).
23 Bratman, S. v. et al. Personalized circulating tumor DNA analysis as a predictive biomarker in solid tumor patients treated with pembrolizumab. Nat Cancer 1, 873-881 (2020).
24 Yang, S. C. et al. Pan-cancer analysis of longitudinal metastatic tumors reveals genomic alterations and immune landscape dynamics associated with pembrolizumab sensitivity. Nat Commun 12, 5137 (2021).
25 Mimitou, E. P. et al. Multiplexed detection of proteins, transcriptomes, clonotypes and CRISPR perturbations in single cells. Nat Methods 16, 409-412 (2019).
26. Stuart, T. et al. Comprehensive Integration of Single-Cell Data. Cell 177, 1888-1902.e21 (2019).
27. Borcherding, N. & Bormann, N. L. scRepertoire: An R-based toolkit for single-cell immune receptor analysis. F1000Res 9, 47 (2020).
28 Borcherding, N. et al. Mapping the immune environment in clear cell renal carcinoma by single-cell genomics. Commun Biol 4, (2021).
29 de Vries, N. L. et al. γδ T cells are effectors of immunotherapy in cancers with HLA class I defects. Nature (2023) doi: 10.1038/s41586-022-05593-1.
30 Rossi, C. et al. Boosting γδ T cell-mediated antibody-dependent cellular cytotoxicity by PD-1 blockade in follicular lymphoma. Oncoimmunology 8, (2019).
31. Nada, M. H., Wang, H., Hussein, A. J., Tanaka, Y. & Morita, C. T. PD-1 checkpoint blockade enhances adoptive immunotherapy by human Vy2V02 T cells against human prostate cancer. Oncoimmunology 10, (2021).
32. Schondelmaier, S., Wesch, D., Pechhold, K. & Kabelitz, D. Vy gene usage in peripheral blood γδ T cells. Immunol Lett 38, 121-6 (1993).
33. Iwasaki, M. et al. Expression and function of PD-1 in human γδ T cells that recognize phosphoantigens. Eur J Immunol 41, 345-355 (2011).
34. Joachims, M. L., Chain, J. L., Hooker, S. W., Knott-Craig, C. J. & Thompson, L. F. Human alpha beta and gamma delta thymocyte development: TCR gene rearrangements, intracellular TCR beta expression, and gamma delta developmental potential—differences between men and mice. J Immunol 176, 1543-52 (2006).
35. Moore, M. J. et al. ZFP36 RNA-binding proteins restrain T cell activation and anti-viral immunity. Elife 7, (2018).
36. Ko, H. S., Fu, S. M., Winchester, R. J., Yu, D. T. & Kunkel, H. G. la determinants on stimulated human T lymphocytes. Occurrence on mitogen- and antigen-activated T cells. J Exp Med 150, 246-55 (1979).
37. Yost, K. E. et al. Clonal replacement of tumor-specific T cells following PD-1 blockade. Nat Med 25, 1251-1259 (2019).
38 Silva-Santos, B., Mensurado, S. & Coffelt, S. B. γδ T cells: pleiotropic immune effectors with therapeutic potential in cancer. Nature Reviews Cancer vol. 19 392-404 Preprint at https://doi.org/10.1038/s41568-019-0153-5 (2019).
39. Gao, Y. et al. γδ T cells provide an early source of interferon γ in tumor immunity. Journal of Experimental Medicine 198, 433-442 (2003).
40. Wakita, D. et al. Tumor-infiltrating IL-17-producing γδ T cells support the progression of tumor by promoting angiogenesis. Eur J Immunol 40, 1927-1937 (2010).
41. Coffelt, S. B. et al. IL-17-producing γδ T cells and neutrophils conspire to promote breast cancer metastasis. Nature 522, 345-348 (2015).
42. Rei, M. et al. Murine CD27 (−) Vy6 (+) γδ T cells producing IL-17A promote ovarian cancer growth via mobilization of protumor small peritoneal macrophages. Proc Natl Acad Sci USA 111, (2014).
43. Wu, Y. et al. An innate-like Vo1+γδ T cell compartment in the human breast is associated with remission in triple-negative breast cancer. Sci Transl Med 11, (2019).
44. Wu, Y. et al. A local human Vo1 T cell population is associated with survival in nonsmall-cell lung cancer. Nat Cancer 3, 696-709 (2022).
45. Foord, E., Arruda, L. C. M., Gaballa, A., Klynning, C. & Uhlin, M. Characterization of ascites- and tumor-infiltrating γδ T cells reveals distinct repertoires and a beneficial role in ovarian cancer. Sci Transl Med 13, 192 (2021).
46. Daley, D. et al. γδ T Cells Support Pancreatic Oncogenesis by Restraining αβ T Cell Activation. Cell 166, 1485-1499.e15 (2016).
47. Wu, P. et al. γδT17 cells promote the accumulation and expansion of myeloid-derived suppressor cells in human colorectal cancer. Immunity 40, 785-800 (2014).
48. Zaretsky, J. M. et al. Mutations Associated with Acquired Resistance to PD-1 Blockade in Melanoma. New England Journal of Medicine 375, 819-829 (2016).
49. McGranahan, N. et al. Allele-Specific HLA Loss and Immune Escape in Lung Cancer Evolution. Cell 171, 1259-1271.e11 (2017).

1. A vector comprising a nucleic acid sequence encoding a T cell receptor heterodimer, or a T cell receptor γ peptide chain capable of forming a functional T cell receptor heterodimer together with a T cell receptor δ peptide chain and/or a T cell receptor δ peptide chain capable of forming a functional T cell receptor heterodimer together with a T cell receptor γ peptide chain, wherein the nucleic acid sequence encodes CATWDGSRSYYKKLF (SEQ ID NO: 3) as complementarity-determining region 3y (CDR3γ) and/or CALGDYVLSDKLIF (SEQ ID NO: 6) as complementarity-determining region 3δ (CDR30).

2. The vector according to claim 1, wherein the vector encodes: a first polypeptide comprising three γ complementarity-determining regions (CDRs) comprising the amino acid sequences: EGSNGY (SEQ ID NO: 1) as CDR1γ, YDSYNSKVVLES (SEQ ID NO: 2) as CDR2γ and CATWDGSRSYYKKLF (SEQ ID NO: 3) as CDR3γ; and/or a second polypeptide comprising three CDRs comprising the amino acid sequences: TSWWSYY (SEQ ID NO: 4) as CDR10, QGSDEQ (SEQ ID NO: 5) as CDR20, and CALGDYVLSDKLIF (SEQ ID NO: 6) as CDR30.

3. A host cell comprising a vector as defined in claim 1.

4. The host cell according to claim 3, wherein the host cell is a T cell and the T cell expresses said T cell receptor.

5. A transduced T cell expressing a T cell receptor heterodimer comprising a first polypeptide comprising three γ CDRs comprising the sequences: EGSNGY (SEQ ID NO: 1) as CDR1Y, YDSYNSKVVLES (SEQ ID NO: 2) as CDR2γ and CATWDGSRSYYKKLF (SEQ ID NO: 3) as CDR3γ and a second polypeptide comprising three CDRs comprising the sequences: TSWWSYY (SEQ ID NO: 4) as CDR1δ, QGSDEQ (SEQ ID NO: 5) as CDR20, and CALGDYVLSDKLIF (SEQ ID NO: 6) as CDR30.

6. The vector according to claim 1, wherein the T cell receptor heterodimer does not bind to cluster of differentiation 1 (CD1) CD1c and monomorphic antigen-presenting molecule MHC-related protein-1 (MR1) expressed on a tumour cell.

7. A population of cells comprising or consisting of T cells as defined in claim 5.

8.-13. (canceled)

14. A bispecific antibody comprising: an anti-CD3 antibody or fragment thereof that binds to a CD3 receptor of a T cell; and a T cell receptor heterodimer as defined in claim 5 or a fragment thereof that binds to a tumor cell.

15. The bispecific antibody of claim 14, further comprising a linking region between the anti-CD3 antibody or fragment and the T cell receptor heterodimer or fragment thereof, optionally an amino acid spacer.

16. An antibody-drug conjugate comprising a T cell receptor heterodimer as defined in claim 5 or a fragment thereof that binds to a tumor cell, conjugated to a cytotoxic cancer treatment.

17. (canceled)

18. A method of treating cancer in a subject comprising administering to the subject a therapeutically effective amount of the population of cells according to claim 7.

19. The method according to claim 18 wherein the cancer is selected from Merkel cell cancer, melanoma, breast cancer, ovarian cancer, myeloma and lung cancer and/or wherein the cancer is a tumor that lacks or has downregulated MHC class I antigen presentation.

20. The method according to claim 18, wherein the subject has been administered an immune checkpoint inhibitor.

21. A method of treating cancer in a subject in need thereof comprising administering a therapeutically effective amount of the antibody-drug conjugate of claim 16 to the subject in need thereof.

22. The method according to claim 21 wherein the cancer is selected from Merkel cell cancer, melanoma, breast cancer, ovarian cancer, myeloma and lung cancer and/or wherein the cancer is a tumor that lacks or has downregulated MHC class I antigen presentation.

23. A method of treating a proliferative disease or an autoimmune disease in a subject in need thereof comprising administering a therapeutically effective amount of the population of cells according to claim 7 to a subject in need thereof.

24. A population of cells comprising or consisting of T cells as defined in claim 1.

25. A method of treating a proliferative disease or an autoimmune disease in a subject in need thereof comprising administering a therapeutically effective amount of the population of cells according to claim 24 to the subject in need thereof.

26. The method of claim 25, wherein the proliferative disease is cancer.

27. The method of claim 26, wherein the cancer is selected from Merkel cell cancer, melanoma, breast cancer, ovarian cancer, myeloma and lung cancer and/or wherein the cancer is a tumor that lacks or has downregulated MHC class I antigen presentation.
